(19) United States
(12) Patent Application Publication
(10) Pub. No.: US 2007/0129578 A1
(43) Pub. Date: Jun. 7, 2007

(54) Method for the production of 2,2'-dihydroxybiphenyls

(75) Inventors: Ralf Widmaier, Mannheim (DE); Bettina Muller, Mannheim (DE); Marianna Pierobon, Ludwigshafen (DE)

Correspondence Address: CONNOLLY BOVE LODGE & HUTZ, LLP, P O BOX 2207, WILMINGTON, DE, 19899, US

(73) Assignee: BASF Aktiengesellschaft, ludwigshafen (DE)

(21) Appl. No.: 10/576,694

(22) PCT Filed: Oct. 21, 2004
(86) PCT No.: PCT/EP04/12117

§ 371 Date: Apr. 21, 2006

(30) Foreign Application Priority Data
Oct. 29, 2003 (DE) 10350817.1

(51) Int. Cl. C07C 39/12

(52) U.S. Cl. 568/747

(57) ABSTRACT

A process for the preparation of a 2,2′-dihydroxybiphenyl by oxidative coupling of two phenyl molecules which have a hydrogen atom in an o-position by means of a peroxide in the presence of water at from 0 to 100° C., wherein the preparation is carried out in the presence of a water-insoluble polymer comprising a) from 0 to less than 100% by weight of a vinyl heterocycle b) from 0 to 10% by weight of a difunctional crosslinking component c) from 0 to less than 100% by weight of styrene or of a monounsaturated styrene derivative or a mixture thereof d) from 0 to 100% by weight of a N-vinylamide of an aliphatic carboxylic acid, or the monomers obtained by hydrolysis of said amido group e) from 0 to 100% by weight of a vinylcarboxylic acid or the esters, amides or salts thereof, or the monomers obtained by hydrolysis of said amido group or ester group, with the proviso that the content of compound d) or e), or d) and e), is more than 0% by weight.

The present invention relates to a process for the preparation of a 2,2′-dihydroxybiphenyl by oxidative coupling of two phenol molecules which have a hydrogen atom in an o-position by means of a peroxide in the presence of water at from 0 to 100° C.

It is known that 2,2′-dihydroxybiphenyls, such as 2,2′-dihydroxy-3,3′,5,5′-tetramethylbiphenyl, can be used for the preparation of bidentate phosphite, bidentate phosphonite and bidentate phosphinite ligands.

It is known that 2,2′-dihydroxybiphenyls can be prepared by oxidative coupling of two phenol molecules which have a hydrogen atom in an o-position by means of a peroxide in the presence of water at from 0 to 100° C.

For instance, U.S. Pat. No. 6,077,979 describes the preparation of 2,2′-dihydroxy-3,3′,5,5′-tetramethylbiphenyl by oxidative coupling of two molecules of 2,4-dimethylphenol by means of persulfate and catalytically active amounts of iron or of an iron compound in the presence of water at from 0 to 100° C.

The problem with this reaction is that a rubber-like, tacky material forms during the reaction and leads to coating of the reactor and of the reactor internals, such as stirrers. Particularly in the procedure on an industrial scale, this means that heat exchange and thorough mixing of the reaction mixture are scarcely possible, complete conversion of the starting materials cannot be achieved and moreover the working-up of the product mixture obtained is extremely difficult.

It is an object of the present invention to provide a process which permits the preparation of a 2,2′-dihydroxybiphenyl by oxidative coupling of two phenol molecules which have a hydrogen atom in an o-position by means of a peroxide in the presence of water at from 0 to 100° C. and the working-up of the resulting product mixture in a technically simple and economical manner avoiding said disadvantages.

We have found that this object is achieved by a process for the preparation of a 2,2′-dihydroxybiphenyl by oxidative coupling of two phenol molecules which have a hydrogen atom in an o-position by means of a peroxide in the presence of water at from 0 to 100° C., wherein the preparation is carried out in the presence of a water-insoluble polymer comprising

a) from 0.1 to 99.9% by weight of at least one vinyl heterocycle,

b) from 0.1 to 10% by weight of at least one difunctional crosslinking component,

c) from 0 to 99.8% by weight of styrene or at least one monounsaturated styrene derivative or a mixture thereof,

the stated percentages by weight of the individual components a), b) and c) summing to 100%.

We have furthermore found a process for the preparation of a 2,2′-dihydroxybiphenyl by oxidative coupling of two phenol molecules which have a hydrogen atom in an o-position by means of a peroxide in the presence of water at from 0 to 100° C., wherein the preparation is carried out in the presence of a water-insoluble polymer comprising
a) from 0 to less than 100% by weight of at least one vinyl heterocycle
b) from 0 to 10% by weight of at least one difunctional crosslinking component
c) from 0 to less than 100% by weight of styrene or at least one monounsaturated styrene derivative or a mixture thereof
d) from 0 to 100% by weight of at least one N-vinylamide of an aliphatic carboxylic acid, or of the monomers obtained by partial or complete hydrolysis of said amido group
e) from 0 to 100% by weight of at least one vinylcarboxylic acid or of esters, amides or salts thereof or of monomers obtained by partial or complete hydrolysis of said amido group or ester group,
   with the proviso that
   the content of compound d) is more than 0% by weight or
   the content of compound e) is more than 0% by weight or
   the content both of compound d) and of compound e) is more than 0% by weight,
   the stated percentages by weight of the individual components a), b), c), d) and e) summing to 100%.

Phenols which have a hydrogen atom in an o-position are generally known and commercially available.

The preparation of one of the preferred starting compounds 2,4-dimethylphenol is known and the compound is commercially available.

In a further preferred embodiment, phenol (hydroxybenzene) can be used as the phenol which has a hydrogen atom in an o-position.

In another preferred embodiment, 2,3,5-trimethylphenol can be used as the phenol which has a hydrogen atom in an o-position.

In a further preferred embodiment, the phenol used may be a 2-alkylphenol, preferably a C₁-C₄-alkylphenol, such as 2-methyl-phenol, 2-ethylphenol, 2-n-propyl-phenol, 2-isopropylphenol, 2-n-butylphenol, 2-tert-butylphenol, 2-isobutylphenol, in particular 2-methylphenol or 2-tert-butylphenol.

In a further preferred embodiment, the phenol used may be a 2,4-dialkylphenol, preferably of the type 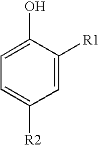
where R1 and R2, independently of one another, are methyl, ethyl, n-propyl, isopropyl, n-butyl, tert-butyl or isobutyl, such as 2,4-dimethylphenol, 2-methyl-4-ethylphenol, 2-methyl-4-n-propylphenol, 2-methyl-4-isopropylphenol, 2-methyl-4-n-butylphenol, 2-methyl-4-tert-butylphenol, 2-methyl-4-isobutylphenol, 2-ethyl-4-methylphenol, 2,4-diethylphenol, 2-ethyl-4-n-propylphenol, 2-ethyl-4-isopropylphenol, 2-ethyl-4-n-butyl-phenol, 2-ethyl-4-tert-butylphenol, 2-ethyl-4-isobutylphenol, 2-n-propyl-4-methylphenol, 2-n-propyl-4-ethylphenol, 2,4-di-n-propylphenol, 2-n-propyl-4-isopropylphenol, 2-n-propyl-4-n-butylphenol, 2-n-propyl-4-tert-butylphenol, 2-n-propyl-4-isobutylphenol, 2-isopropyl-4-methylphenol, 2-isopropyl-4-ethylphenol, 2-isopropyl-4-n-propylphenol, 2,4-diisopropylphenol, 2-isopropyl-4-n-butylphenol, 2-isopropyl-4-tert-butylphenol, 2-isopropyl-4-isobutylphenol, 2-n-butyl-4-methylphenol, 2-n-butyl-4-ethylphenol, 2-n-butyl-4-n-propylphenol, 2-n-butyl-4-isopropylphenol, 2,4-di-n-butylphenol, 2-n-butyl-4-tert-butylphenol, 2-n-butyl-4-isobutylphenol, 2-tert-butyl-4-methylphenol, 2-tert-butyl-4-ethylphenol, 2-tert-butyl-4-n-propylphenol, 2-tert-butyl-4-isopropylphenol, 2-tert-butyl-4-n-butylphenol, 2,4-di-tert-butylphenol, 2-tert-butyl-4-isobutylphenol, 2-isobutyl-4-methylphenol, 2-isobutyl-4-ethylphenol, 2-isobutyl-4-n-propylphenol, 2-isobutyl-4-isopropylphenol, 2-isobutyl-4-n-butylphenol, 2-isobutyl-4-tert-butylphenol, 2,4-diisobutylphenol, in particular 2,4-dimethylphenol or 2,4-di-tert-butylphenol, particularly preferably 2,4-dimethylphenol.

In a further preferred embodiment, the phenol used may be a 2,5-dialkylphenol, preferably of the type 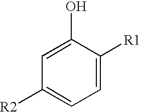
where R1 and R2, independently of one another, are methyl, ethyl, n-propyl, isopropyl, n-butyl, tert-butyl or isobutyl, such as 2,5-dimethylphenol, 2-methyl-5-ethylphenol, 2-methyl-5-n-propylphenol, 2-methyl-5-isopropylphenol, 2-methyl-5-n-butylphenol, 2-methyl-b-tert-butylphenol, 2-methyl-5-isobutylphenol, 2-ethyl-5-methylphenol, 2,5-diethylphenol, 2-ethyl-5-n-propylphenol, 2-ethyl-5-isopropylphenol, 2-ethyl-5-n-butylphenol, 2-ethyl-5-tert-butylphenol, 2-ethyl-5-isobutylphenol, 2-n-propyl-5-methylphenol, 2-n-propyl-5-ethylphenol, 2,5-di-n-propylphenol, 2-n-propyl-5-isopropylphenol, 2-n-propyl-5-n-butylphenol, 2-n-propyl-5-tert-butylphenol, 2-n-propyl-5-isobutylphenol, 2-isopropyl-5-methylphenol, 2-isopropyl-5-ethylphenol, 2-isopropyl-5-n-propylphenol, 2,5-diisopropylphenol, 2-isopropyl-5-n-butylphenol, 2-isopropyl-5-tert-butylphenol, 2-isopropyl-5-isobutylphenol, 2-n-butyl-5-methylphenol, 2-n-butyl-5-ethylphenol, 2-n-butyl-5-n-propylphenol, 2-n-butyl-5-isopropylphenol, 2,5-di-n-butylphenol, 2-n-butyl-5-tert-butylphenol, 2-n-butyl-5-isobutylphenol, 2-tert-butyl-5-methylphenol, 2-tert-butyl-5-ethylphenol, 2-tert-butyl-5-n-propylphenol, 2-tert-butyl-5-isopropylphenol, 2-tert-butyl-5-n-butylphenol, 2,5-di-tert-butylphenol, 2-tert-butyl-5-isobutylphenol, 2-isobutyl-5-methylphenol, 2-isobutyl-5-ethylphenol, 2-isobutyl-5-n-propylphenol, 2-isobutyl-5-isopropylphenol, 2-isobutyl-5-n-butylphenol, 2-isobutyl-5-tert-butylphenol, 2,5-diisobutylphenol, in particular 2-isopropyl-5-methylphenol.

In a further preferred embodiment, the phenol used may be a 3,5-dialkylphenol, preferably of the type 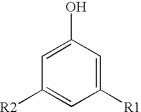
where R1 and R2, independently of one another, are methyl, ethyl, n-propyl, isopropyl, n-butyl, tert-butyl or -isobutyl, such as 3,5-dimethylphenol, 2-methyl-5-ethylphenol, 3-methyl-5-n-propylphenol, 3-methyl-5-isopropylphenol, 3-methyl-5-n-butylphenol, 3-methyl-5-tert-butylphenol, 3-methyl-5-isobutylphenol, 3-ethyl-5-methylphenol, 3,5-diethylphenol, 3-ethyl-5-n-propylphenol, 3-ethyl-5-isopropylphenol, 3-ethyl-5-n-butylphenol, 3-ethyl-5-tert-butylphenol, 3-ethyl-5-isobutylphenol, 3-n-propyl-5-methylphenol, 3-n-propyl-5-ethylphenol, 3,5-di-n-propylphenol, 3-n-propyl-5-isopropylphenol, 3-n-propyl-5-n-butylphenol, 3-n-propyl-5-tert-butylphenol, 3-n-propyl-5-isobutylphenol, 3-isopropyl-5-methylphenol, 3-isopropyl-5-ethylphenol, 3-isopropyl-5-n-propylphenol, 3,5-diisopropylphenol, 3-isopropyl-5-n-butylphenol, 3-isopropyl-5-tert-butylphenol, 3-isopropyl-5-isobutylphenol, 3-n-butyl-5-methylphenol, 3-n-butyl-5-ethylphenol, 3-n-butyl-5-n-propylphenol, 3-n-butyl-5-isopropylphenol, 3,5-di-n-butylphenol, 3-n-butyl-5-t-butylphenol, 3-n-butyl-5-isobutylphenol, 3-tert-butyl-5-methylphenol, 3-tert-butyl-5-ethylphenol, 3-tert-butyl-5-n-propylphenol, 3-tert-butyl-5-isopropylphenol, 3-tert-butyl-5-n-butylphenol, 3,5-di-tert-butylphenol, 3-tert-butyl-5-isobutylphenol, 3-isobutyl-5-methylphenol, 3-isobutyl-5-ethylphenol, 3-isobutyl-5-n-propylphenol, 3-isobutyl-5-isopropylphenol, 3-isobutyl-5-n-butylphenol, 3-isobutyl-5-tert-butylphenol, 3,5-diisobutylphenol, in particular 3,5-dimethylphenol 3,5-di-tert-butylphenol.

In a further preferred embodiment, the phenol used may be a 2,3,5-trialkylphenol, preferably of the type 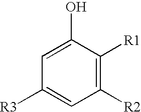
where R1, R2 and R3, independently of one another, are methyl, ethyl, n-propyl, isopropyl, n-butyl, tert-butyl or isobutyl, in particular 2,3,5-trimethylphenol.

The use of 2,4-dimethylphenol as the phenol is particularly preferred. According to the invention, the 2,2′-dihydroxybiphenyl obtained is 2,2′-dihydroxy-3,3′,5,5′-tetramethylbiphenyl.

It is also possible to use mixtures of the phenols, in particular of said phenols.

Advantageously, only one phenol is used.

The novel process is particularly advantageous in the case of those phenols which are present in liquid form at the chosen reaction temperature and whose 2,2′-dihydroxybiphenyl obtained in a corresponding manner by coupling are present in solid form at this temperature.

The use of 2,4-dimethylphenol as the phenol in the reaction at 20-50° C. to give 2,2′-dihydroxy-3,3′,5,5′-tetramethylbiphenyl as the 2,2′-dihydroxybiphenyl is particularly preferred.

If phenols which are present in solid form at the chosen reaction temperature are used, it has proven advantageous to dissolve the phenol in a liquid diluent, preferably an organic liquid diluent, in particular hexane, to add to the mixture an insoluble polymer comprising

a) from 0.1 to 99.9% by weight of at least one vinyl heterocycle

b) from 0.1 to 10% by weight of at least one difunctional crosslinking component

c) from 0 to 99.8% by weight of styrene or at least one monounsaturated styrene derivative or a mixture thereof,

the stated percentages by weight of the individual components a), b) and c) summing to 100%, to remove the liquid diluent, preferably by distillation, and to add the product obtained to the reaction mixture for the preparation of a 2,2′-dihydroxybiphenyl.

If phenols which are present in solid form at the chosen reaction temperature are used, it has furthermore proven advantageous to dissolve the phenol in a liquid diluent, preferably an organic liquid diluent, in particular hexane, to add to the mixture an insoluble polymer comprising
a) from 0 to less than 100% by weight of at least one vinyl heterocycle
b) from 0 to 10% by weight of at least one difunctional crosslinking component
c) from 0 to less than 100% by weight of styrene or at least one monounsaturated styrene derivative or a mixture thereof
d) from 0 to 100% by weight of at least one vinylamide of an aliphatic carboxylic acid, or the monomers obtained by partial or complete hydrolysis of said amido group
e) from 0 to 100% by weight of at least one vinylcarboxylic acid or the esters, amides or salts thereof or the monomers obtained by partial or complete hydrolysis of said amido group or ester group,
f) with the proviso that
g) the content of compound d) is more than 0% by weight or
h) the content of compound e) is more than 0% by weight or
i) the content both of compound d) and of compound e) is more than 0% by weight,
   the stated percentages by weight of the individual components a), b), c), d) and e) summing to 100%,
   to remove the liquid diluent, preferably by distillation, and to add the product obtained to the reaction mixture for the preparation of a 2,2′-dihydroxybiphenyl.

According to the invention, the reaction is carried out at from 0 to 100° C. In an advantageous embodiment, temperatures of not more than 50° C. are suitable. In an advantageous embodiment, temperatures of at least 15° C. are suitable. Particularly preferably, the process is carried out at from 15 to 50° C.

According to the invention, the coupling reaction is carried out by means of a peroxide. A suitable peroxide is an organic, preferably inorganic, peroxide, it being possible to use one peroxide or a mixture of peroxides.

The organic peroxide used may advantageously be a compound of the formula (tert-butyl)-O—O—R where R is H, tert-butyl or a mixture thereof, in particular di-tert-butyl peroxide.

The inorganic peroxide used can preferably be a persulfate anion, in particular in the form of a salt which has at least slight solubility in water. Preferred persulfates are those which contain an ammonium, sodium or potassium cation or a mixture thereof as the cation. Accordingly, the persulfate anion is preferably used in the presence of an ammonium, sodium or potassium cation.

The inorganic peroxide used may be hydrogen peroxide, in particular together with persulfate anion, preferably in an aqueous solution containing from 3 to 55% by weight of hydrogen peroxide.

To achieve a high conversion, the addition of 0.5 mol of peroxide units reactive for the coupling per mole of phenol is theoretically required. The addition of an amount of from 0.5 to 1.2, in particular from 0.6 to 0.8, mol of peroxide units reactive for the coupling per mole of phenol has proven advantageous.

Processes for the preparation of such peroxides are known per se, and the peroxides are commercially available.

In a preferred embodiment, the novel reaction can be carried out in the presence of amounts of a metal, preferably iron, cobalt, copper, manganese, titanium, silver, palladium, particularly preferably iron, copper, in particular iron, which are suitable as a catalyst, or of a compound of such a metal. In the context of the present invention, a metal is understood as meaning a metal or a mixture of a plurality of metals, and a compound of such a metal is understood as meaning one or more compounds of one or more metals.

If iron is used, it can advantageously be employed in the form of an iron-containing alloy, from which iron is dissolved out in the reaction mixture. If iron is used in the form of an iron compound, the iron may be present in divalent or trivalent form. Here, the iron may be used as an organic or, preferably, inorganic compound, in particular as a salt, for example as the sulfate or chloride.

It is possible that the water-soluble polymer will form complexes with the metal or the metal compound. This complex formation can positively influence the coupling reaction.

The amount of iron used is not critical per se. Preferably from 0.01 to 0.10, in particular from 0.02 to 0.05, mol of iron per mole of phenol can be used.

According to the invention, the reaction is carried out in the presence of a water-insoluble polymer comprising

a) from 0.1 to 99.9% by weight of at least one vinyl heterocycle

b) from 0.1 to 10% by weight of at least one difunctional crosslinking component

c) from 0 to 99.8% by weight of styrene or at least one monounsaturated styrene derivative or a mixture thereof,

the stated percentages by weight of the individual components a), b) and c) summing to 100%.

The vinyl heterocycle used can preferably be at least one N-vinyllactam or at least one N-vinylamine or a mixture thereof.

An advantageous N-vinyllactam is N-vinylpyrrolidone(1-vinyl-2-pyrrolidone), N-vinylpiperidone, N-vinylcaprolactam or a mixture thereof. It is also possible to use substituted N-vinyllactams, as described in U.S. Pat. No. 3,878,310, columns 3-4. Such substituted N-vinyllactams are, for example, N-vinyl-substituted derivatives of the following lactams: 3,3-dimethyl-2-pyrrolidone, 4,4-dimethyl-2-pyrrolidone, 3,4-dimethyl-2-pyrrolidone, 3-ethyl-2-pyrrolidone, 3,5-dimethyl-2-pyrrolidone, 3-phenyl-2-pyrrolidone, 4-acryloyl-2-pyrrolidone, 5-ethyl-2-pyrrolidone, 3-methyl-2-pyrrolidone, 4-methyl-2-pyrrolidone, 5-methyl-2-pyrrolidone, 3,3,5-trimethyl-2-pyrrolidone; 2-piperidone, 5,5-diethyl-2-piperidone, 5,6-dimethyl-2-piperidone, 4-ethyl-2-piperidone, 6-ethyl-2-piperidone, 6-ethyl-3-methyl-2-piperidone, 3-methyl-2-piperidone, 4-methyl-2-piperidone, 5-methyl-2-piperidone, 6-methyl-2-piperidone; 2-caprolactam, 3,6-dimethyl-2-caprolactam, 4,6-dimethyl-2-caprolactam, 7,7-diethyl-2-caprolactam, 3-ethyl-2-caprolactam, 5-ethyl-2-caprolactam, 6-ethyl-2-caprolactam, 7-ethyl-2-caprolactam, 4-ethyl-6-methyl-2-caprolactam, 6-ethyl-4-methyl-2-caprolactam, 3-methyl-2-caprolactam, 4-methyl-2-caprolactam, 5-methyl-2-caprolactam, 6-methyl-2-caprolactam; 2-oxazinidinone (cf. U.S. Pat. Nos. 2,905,669 and 3 097 087), 5-ethyl-2-oxazinidinone, 5-phenyl-2-oxazinidinone, 4,5-dimethyl-2-oxazinidinone, 5,5-dimethyl-2-oxazinidinone, 2,5-diphenyl-2-oxazinidinone, 2-phenyl-4-oxothiazolidone, 2,2′-diphenyl-4-oxothiazolidone, 2,2′-dimethyl-4-oxothiazolidone; 2-oxazolidinone (cf. U.S. Pat. Nos. 2,905,690 and 2 891 058), 5-methyl-2-oxazolidinone, 4-methyl-2-oxazolidinone, 5-ethyl-2-oxazolidinone, 4,5-dimethyl-2-oxazolidone, 2-phenyl-2-oxazolidinone, 5-butyl-2-oxazolidinone, 5-propyl-2-oxazolidinone, 4,5-diethyl-2-oxazolidinone; 3-morpholinones (cf. U.S. Pat. No. 2,987,509), e.g. 5-methyl-3-morpholinone, 5-ethyl-3-morpholinone, 3,5-dimethyl-3-morpholinone; 2-piperazinones (cf. J. Am. Chem. Soc. 62 (1940), 1202); 3,3-dimethyl-2-ketopiperazine, 3-methyl-2-ketopiperazine; 4-thiazolidone (cf. J. Am. Chem. Soc, 76 (1954), 578), 2-methyl-4-thiazolidinone, 2-phenyl-4-thiazolidinone, 2-phenyl-4-thiazolidinone dioxide; 2-thiazolidone (cf. J. Chem. Soc. (1949), 2367), 3-thiamorpholinone; 2-pyrimidone (cf. e.g. J. Chem. Soc. (1959), 525); 2-imidazolidones (cf. e.g. Ann. 232 (1885), 1222)); N,N-ethylenethioureas (cf. e.g. J. Biol. Chem. 163 (1946), 761); tetrahydro-[2H,1,3]-oxazin-3-ones, (cf. e.g. U.S. Pat. No. 2,940,971), and other lactams.

Vinylimidazole, N-vinyl-2-methylimidazole, N-vinyl-4-methylimidazole or mixtures thereof can preferably be used as the N-vinylamine.

Suitable difunctional crosslinking components are compounds which contain at least two ethylenically unsaturated, nonconjugated double bonds in the molecule. Preferred crosslinking agents are N—N′-dialkyleneureas, and in addition alkylenebisacrylamides and alkylene glycol diacrylates or dimethacrylates. Triallylamine, and the crosslinking agents mentioned in column 6 of U.S. Pat. No. 3,878,310 and on page 4 of DE-A 42 37 439, are also suitable. Suitable crosslinking agents are, for example, N,N′-methylenebisacrylamide, N,N′-acryloylethylenediamine, N,N′-divinylethyleneurea, N.N′-divinylpropyleneurea, the alkylene glycol diacrylates and dimethacrylates, e.g. ethylene glycol di(meth)acrylate, diethylene glycol di(meth)acrylate, tetraethylene glycol di(meth)acrylate, higher molecular weight polyethylene glycol dimethacrylates, 1,3- and 1,4-butanediol diacrylates and dimethacrylates, and furthermore divinyl aromatic compounds, such as divinylbenzene, divinylethylbenzene, divinylchlorobenzene, divinyltoluene, divinylnaphthalene, etc. Other suitable crosslinking agents are, for example, vinyl acrylate, allyl (meth)acrylate, p-isopropenyistyrene, trivinyl meseate, diallyl maleate, divinyl ether, divinyldioxane, 1,3- and 1,4-divinyloxybutane, trivinyl citrate, divinyl o-phenylene diacetates, vinyl allyl ether, diethylene glycol diallyl ether, trivinyl glyceryl ether, divinyl glyceryl ether, pentaerythrityl triallyl ether, tetravinyl pentaerythrityl ether, hexahydro-1,3,5-triacryl-s-triazine, ethylidenebis-3-(N-vinylpyrrolidone), N,N′-divinyldiimidazolyl-(2,2′)-butane, 1,1′-bis(3,3′-vinylbenzimidazolith-2-one)-1,4-butane and dimers of vinylpyrrolidone (cf. U.S. Pat. No. 3,252,995). The notation “ . . . (meth)acrylate” is short for . . . acrylate or . . . methacrylate. Of course, mixtures of a plurality of crosslinking agents may also be used.

The difunctional crosslinking component can also be prepared in situ during the preparation of popcorn polymers, as described in U.S. Pat. No. 5,286,826.

A suitable monounsaturated styrene derivative is, for example, α-methylstyrene, p-methylstyrene, ethylstyrene, tert-butylstyrene, vinyltoluene, 1,1-diphenylethylene or a mixture thereof.

In a particularly preferred embodiment, a water-insoluble polymer comprising

a) from 0.1 to 99.9% by weight of N-vinylpyrrolidone as the vinyl heterocycle

b) from 0.1 to 10% by weight of at least one difunctional crosslinking component

c) from 0 to 99.8% by weight of styrene or at least one monounsaturated styrene derivative or a mixture thereof,

the stated percentages by weight of the individual components a), b) and c) summing to 100%

can be used.

Such polymers and their preparation are known per se, for example from DE-A-199 20 944, EP-A-0177812, EP-A-0088964, EP-A-0351363, WO 02/32544, EP-A-1219349, EP-A-1234608, PCT Application No. PCT/EP03/03439 and EP Application No. 02027072.4.

Preferably suitable polymers are commercially available, for example under the brand name Divergan® RS, Divergan® F, Divergan® EF or Divergan® HM from BASF Aktiengesellschaft, Ludwigshafen/Rhein, Germany or under the brand name Polydor® 10, Polyelar® Super R, Polyclar® V, Polyclar® VT, Polyplasdone® XL, Polyplasdone® XL-10, Agrimer® AT or Agrimer® ATF from International Speciality Products (ISP), Wayne, N.J., USA.

In a further embodiment, the reaction is carried out in the presence of a water-insoluble polymer comprising
a) from 0 to less than 100% by weight of at least one vinyl heterocycle
b) from 0 to 10% by weight of at least one difunctional crosslinking component
c) from 0 to less than 100% by weight of styrene or at least one monounsaturated styrene derivative or a mixture thereof,
d) from 0 to 100% by weight of at least one vinylamide of an aliphatic carboxylic acid or the monomers obtained by partial or complete hydrolysis of said amido group,
e) from 0 to 100% by weight of at least one vinylcarboxylic acid or the esters, amides or salts thereof or the monomers obtained by partial or complete hydrolysis of said amido group or ester group,
f) with the proviso that the content of compound d) is more than 0% by weight or
g) the content of compound e) is more than 0% by weight or
h) the content both of compound d) and of compound e) is more than 0% by weight,
i) the stated percentages by weight of the individual components a), b), c), d) and e) summing to 100%.

At least one N-vinyllactam or at least one N-vinylamidine or a mixture thereof can preferably be used as the vinyl heterocycle.

The N-vinyllactam is advantageously N-vinylpyrrolidone, N-vinylpiperidone, N-vinylcaprolactam or a mixture thereof.

The N-vinylamidines are obtainable by partial or complete hydrolysis of polymers which contain N-vinylcarboxamides, for example N-vinylformamide or derivatives thereof, and subsequent reaction with compounds containing carbonyl groups: the partially hydrolyzed polymers can form intra- and intermolecular amidines by reaction of a polymer-bound amino group with a carbonyl group (a formamide group) from the polymer. As a result of their hydrolysis and subsequent reaction, cyclic amidines form. Acyclic amidines can also be prepared by reaction of amines in partially or completely hydrolyzed polymers of N-vinylcarboxamides, with monomeric, oligomeric or polymeric aldehydes, ketones or formic acid or derivatives thereof. Acyclic amidines are also obtainable by reaction of unhydrolyzed or only partially hydrolyzed polymers of N-vinylcarboxamides with monomeric, oligomeric or polymeric amines, diamines, triamines, etc.

A preferably used N-vinylamidine is N-vinylimidazole, N-vinyl-2-methylimidazole, N-vinyl-4-methylimidazole or a mixture thereof.

In a further embodiment, a water-insoluble polymer comprising

d) 100% by weight of at least one N-vinylamide of an aliphatic carboxylic acid, or the monomers obtained by partial or complete hydrolysis of said amido group and of the formula H₂C═CR²NR³C(O)R¹, where R¹, R² and R³, independently of one another, are hydrogen or C₁₋₂₀-alkyl, C₁₋₂₀-aryl or C₁₋₂₀-alkylaryl,

is used.

An advantageously used N-vinylamide is an N-vinylamide of a carboxylic acid of one to four carbon atoms, such as formic acid, acetic acid, propionic acid, butyric acid or isobutyric acid, preferably formic acid, or a mixture of such vinylamides. Olefinic amines in which plural carbons or preferably one of the two carbons of the olefinic double bond carry a plurality of amino groups, preferably one amino group, can be used as the amide component in such a vinylamide. The olefin can advantageously carry two to six carbon atoms, in particular two carbon atoms. In a particularly preferred embodiment, a suitable amide component is vinylamine.

N-Vinylformamide, N-vinylacetamide, N-vinyl-N-methylacetamide, N-vinyl-N-methylformamide, acyclic amidines or mixtures thereof are preferably used as the N-vinylamide. In a particularly preferred embodiment, N-vinylformamide can be used as the N-vinylamide.

In a preferred embodiment, the amido group of the vinylamide can be partly or completely hydrolyzed before or, preferably, after the preparation of the polymer, to give a polymer having amino groups bonded to the polymer main chain.

If the vinylamide used is N-vinylformamide, polyvinylamine can advantageously be obtained. Accordingly, polyvinylamine can be used as component d). It is not obtainable by polymerization of the (unstable) vinylamine but, as described above, by polymerization of N-vinylformamide and subsequent hydrolysis of the poly-N-vinylformamide formed.

Such polymers and their preparation are known per se. The hydrolysis can be carried out under acidic or basic conditions. Further details of the hydrolysis and the compounds obtainable thereby are given in EP-A 71 050, which is hereby incorporated by reference.

In a further embodiment, a water-insoluble polymer comprising

e) 100% by weight of at least one vinylcarboxylic acid or the esters, amides or salts thereof or the monomers obtained by partial or complete hydrolysis of said amido group or ester group

is used.

In the context of the present invention, a vinylcarboxylic acid is understood as meaning an organic acid in which one or more carboxyl groups are linked to one or more carbon atoms of an olefinic double bond.

Vinylcarboxylic acids of 4 to 6 carbon atoms, preferably acrylic acid, methacrylic acid, crotonic acid, maleic acid, fumaric acid, citraconic acid, mesaconic acid or itaconic acid, in particular acrylic acid, methacrylic acid and maleic acid, are advantageously suitable as at least one vinylcarboxylic acid or the esters, amides or salts thereof or the monomers obtained by partial or complete hydrolysis of said amido group or ester group.

According to the invention, some or all of the carboxyl groups may be present in derivatized form, preferably in the form of the ester group, amido group or as a salt.

Advantageous esters are the esters with an aliphatic alcohol, preferably with an aliphatic alcohol of one to four carbon atoms, such as methanol, ethanol, n-propanol, isopropanol, n-butanol, isobutanol, sec-butanol or tert-butanol, in particular ethanol.

In the amido group, the amide nitrogen can be substituted, for example by one or two aliphatic groups, or preferably unsubstituted.

Furthermore, some or all of the carboxyl groups may be present as a salt, in particular as an alkali metal or alkaline earth metal salt, preferably as the sodium, potassium, magnesium or calcium salt.

Particularly preferred vinylcarboxylic acids or the esters, amides or salts thereof or the monomers obtained by partial or complete hydrolysis of said amido group or ester group are acrylic acid, acrylamide, C₁-C₄-alkyl acrylates, alkali metal acrylate, alkaline earth metal acrylate, methacrylic acid, methacrylamide, N-isopropylmethacrylamide, C₁-C₄-alkyl methacrylates, alkali metal methacrylate, alkaline earth metal methacrylate, maleic acid, maleic anhydride, maleimide, mono(C₁-C₄-alkyl)maleate, di(C₁-C₄-alkyl)maleate, monoalkali metal maleate, dialkali metal maleate, fumaric acid, fumaric acid monoamide, fumaric acid diamide, mono(C₁-C₄-alkyl)fumarate, di(C₁-C₄-alkyl)fumarate, monoalkali metal fumarate, dialkali metal fumarate or mixtures thereof.

Such polymers and their preparation are known per se.

In a further, preferred embodiment, the novel preparation of the 2,2′-dihydroxybiphenyl is carried out in the presence of a polymer blend which comprises
α) a thermoplastic polymer and
β) water-insoluble polymers, preferably those of vinyl heterocycles or N-vinylamides, it being possible for the N-vinylamide polymer to be partly or completely hydrolyzed to amines.

The polymer blend preferably comprises
α) from 1 to 99, preferably from 5 to 95, % by weight of at least one thermoplastic polymer and
β) from 1 to 99, preferably from 1 to 95, % by weight of a polymer selected from poly-N-vinylpyrrolidone, poly-N-vinylformamide and polyamines.

The polyamines can be obtained by partial or complete hydrolysis of the amides, for example of poly-N-vinylformamide, as described above.

Thermoplastic polymers are to be understood as meaning amorphous uncrosslinked and semicrystalline uncrosslinked polymers. They are fusible and can be processed by extrusion, injection molding or a spinning method. They are often soluble in organic solvents. They contain both crystalline and amorphous regions. The macromolecular chains pass through a plurality of regions and are thus responsible for the cohesion of the polymer (cf. for example A. Echte, Handbuch der Technischen Polymerchemie, 1st Edition, 1993, VCH, Weinheim). Suitable thermoplastic polymers are, for example, homo- and copolymers of styrene, polyolefins, vinyl polymers, polyamides, polyesters, polyacetates, polycarbonates or polyurethanes and isomers. For example, semicrystalline thermoplastics, such as polyethylene, polyoxymethylene or polypropylene, or amorphous thermoplastics, such as polyvinyl chloride or polymethacrylate, are suitable.

Particularly preferred thermoplastic polymers are the homo- and copolymers of styrene or of other vinylaromatic compounds. Such polymers are referred to below collectively as styrene polymers. They may be substituted by organic radicals, such as alkyl, aryl, alkylaryl, cycloalkyl or alkoxy, and/or may contain functional groups, for example basic groups, such as amino groups, acidic groups, such as sulfo groups, and the conjugates thereof, such as ammonium, sulfonate or carboxylate groups. The functional groups may be present on the aromatic styrene ring and/or on the radicals.

The styrene polymers include homopolystyrene, copolymers of styrene with comonomers such as acrylonitrile, α-methylstyrene, maleic anhydride, methyl methacrylate and other conventional comonomers, and rubbery (impact-modified) styrene polymers. Such rubbery styrene polymers are, for example, HIPS (high impact polystyrene, polystyrene modified with butadiene rubber), ABS (acrylonitrile/butadiene/styrene polymer) or ASA (acrylonitrile/styrene/acrylate polymer).

The styrene polymers may contain conventional crosslinking monomers, usually in amounts of from 0 to 10% by weight. Such crosslinking agents have been mentioned further above. Furthermore, the divinyl esters of dicarboxylic acids, such as succinic acid and adipic acid, the diallyl ethers and divinyl ethers of bifunctional alcohols, such as of ethylene glycol and of butane-1,4-diol, the diesters of acrylic acid and methacrylic acid with these bifunctional alcohols, triallyl cyanurate, dihydrodicyclopentadienyl acrylate and the allyl esters of acrylic acid or methacrylic acid are suitable as crosslinking agents.

The polymers of vinyl heterocycles or N-vinylamides also include copolymers of a vinyl heterocycle and comonomers or of N-vinylamide and comonomers, in addition to the homopolymers. In particular, poly-N-vinylpyrrolidone includes homo- and copolymers of N-vinylpyrrolidone, and poly-N-vinylformamide includes homo- and copolymers of N-vinylformamide. Suitable comonomers have been mentioned above.

In further, preferred embodiments, the novel preparation of the 2,2′-dihydroxybiphenyls is carried out in the presence of polymer blends which are described below.

In a preferred embodiment, the preparation of the novel biphenyls is carried out in the presence of a polymer blend which comprises
i) from 5 to 95, preferably from 20 to 95, particularly preferably from 50 to 95, % by weight of at least one styrene polymer and
ii) from 5 to 95, preferably from 5 to 80, particularly preferably from 5 to 50, % by weight of a crosslinked or uncrosslinked poly-N-vinylpyrrolidone.

In a likewise preferred embodiment, the preparation is carried out in the presence of a polymer blend which comprises
i) from 5 to 95, preferably from 20 to 95, particularly preferably from 50 to 95, % by weight of at least one styrene polymer and
iii) from 5 to 95, preferably from 5 to 80, particularly preferably from 5 to 50, % by weight of a poly-N-vinylformamide or of a polyamine obtained by partial or complete hydrolysis of the poly-N-vinylformamide.

In a further embodiment, the novel biphenyls are prepared in the presence of a polymer blend which comprises a styrene polymer, poly-N-vinylpyrrolidone and poly-N-vinylformamide (or a polyamine obtained by hydrolysis of the poly-N-vinylformamide).

Particularly preferably, the preparation is carried out in the presence of a polymer blend which comprises
i) from 2 to 95, preferably from 20 to 95, % by weight of at least one styrene polymer,
ii) from 2 to 95, preferably from 3 to 77, % by weight of a crosslinked or uncrosslinked poly-N-vinylpyrrolidone and
iii) from 2 to 95, preferably from 3 to 77, % by weight of a poly-N-vinylformamide or of a polyamine obtained by partial or complete hydrolysis of the poly-N-vinylformamide,
iv) the stated percentages by weight of the individual components i), ii) and iii) summing to 100%.

In a further, preferred embodiment, the novel preparation of the 2,2′-dihydroxybiphenyls is carried out in the presence of a polymer blend which comprises ii) a crosslinked or uncrosslinked poly-N-vinylpyrrolidone and iii) a poly-N-vinylformamide, or the polyamine obtained therefrom by partial or complete hydrolysis.

Particularly preferably, such a polymer blend comprises
ii) from 5 to 95% by weight of a crosslinked or uncrosslinked poly-N-vinylpyrollidone and
iii) from 5 to 95% by weight of a poly-N-vinylformamide or of a polyamine obtained by partial or complete hydrolysis of the poly-N-vinylformamide.

Of course, in the case of all said blends, the stated percentages by weight of the individual components, for example a), b), c), d), e), α), β, i), ii) or iii), sum in each case to 100%.

Said polymer blends are prepared in a manner known per se, for example by extrusion or other conventional mixing methods.

The novel coupling of the phenol molecules can be carried out substantially by processes known per se, for example as described in U.S. Pat. No. 6,077,979. The water-insoluble polymers can be added to the reaction mixture before, during or after the coupling reaction, the addition before the reaction being preferred.

The amount of phenol, based on polymer, can be optimally chosen in various ranges depending on the manner in which the coupling reaction is carried out. If the phenol to be reacted is added completely before the coupling reaction, an amount of phenol of from 10 to 100 mmol, based on 1 g of polymer, has proven advantageous. If the phenol is added gradually to the reaction mixture during the coupling reaction, an amount of phenol of from 50 to 150 mmol, based on 1 g of polymer, has proven advantageous.

After the novel reaction, the product can be separated from the reaction mixture by methods known per se, such as by filtration or extrusion.

For example, the reaction mixture can be filtered to give a filtrate and a filter cake. The product can then advantageously be separated from the filter cake by extraction, preferably with an aliphatic, in particular aromatic, hydrocarbon, such as benzene, o-xylene, m-xylene, p-xylene, in particular toluene. The product can then be obtained from the resulting solution, for example by crystallization or by distilling off the extraction medium.

Furthermore, the product can be extracted from the reaction mixture, preferably with an aliphatic, in particular aromatic, hydrocarbon, such as benzene, o-xylene, m-xylene, p-xylene, in particular toluene. The product can then be obtained from the resulting solution, for example by crystallization or by distilling off the extraction medium.

### EXAMPLES

### Examples 1-5

0.7 g (2.5 mmol) of FeSO₄.7H₂O in 75 ml of water was initially taken in a 250 ml flask which carried a thermometer and was equipped with a Teflon paddle stirrer (350 revolutions per minute), and 1.3 g of divergan® RS (from BASF Aktiengesellschaft, Ludwigshafen/Rhein, Germany) were added. The suspension was

[symbol]

1. A process for the preparation of a 2,2′-dihydroxybiphenyl comprising oxidatively coupling two phenol molecules which have a hydrogen atom in an o-position in the presence of a peroxide and water at from 0 to 100° C., wherein the preparation is conducted in the presence of a water-insoluble polymer, comprising
a) from 0.1 to 99.9% by weight of at least one vinyl heterocycle
b) from 0.1 to 10% by weight of at least one difunctional crosslinking component
c) from 0 to 99.8% by weight of styrene or at least one monounsaturated styrene derivative or a mixture thereof, the stated percentages by weight of the individual components a), b) and c) summing to 100%.

2. A process for the preparation of a 2,2′-dihydroxybiphenyl comprising oxidatively coupling two phenol molecules which have a hydrogen atom in an o-position in the presence of a peroxide and water at from 0 to 100° C., wherein the preparation is conducted in the presence of a water-insoluble polymer, comprising
a) from 0 to less than 100% by weight of at least one vinyl heterocycle
b) from 0 to 10% by weight of at least one difunctional crosslinking component
c) from 0 to less than 100% by weight of styrene or at least one monounsaturated styrene derivative or a mixture thereof
d) from 0 to 100% by weight of at least one N-vinylamide of an aliphatic carboxylic acid, or of the monomers obtained by partial or complete hydrolysis of said amido group
e) from 0 to 100% by weight of at least one vinylcarboxylic acid or its esters, amides or salts or of the monomers obtained by partial or complete hydrolysis of said amido group or ester group, with the proviso that
the content of compound d) is more than 0% by weight or
the content of compound e) is more than 0% by weight or
the content both of compound d) and of compound e) is more than 0% by weight, the stated percentages by weight of the individual components a), b), c), d) and e) summing to 100%.

3. A process according to claim 1, the process being carried out at from 15 to 50° C.

4. A process according to claim 3, wherein the peroxide is an inorganic peroxide.

5. A process according to claim 3, wherein the peroxide is a persulfate anion.

6. A process according to claim 5, wherein the persulfate anion comprises an ammonium, sodium or potassium cation.

7. A process according to claim 1, wherein the coupling is conducted in the presence of iron or of an iron compound which are suitable as catalyst.

8. A process according to claim 1, wherein the phenol is 2,4-dimethylphenol, and the 2,2′-dihydroxybiphenyl obtained is 2,2′-dihydroxy-3,3′,5,5′-tetramethylbiphenyl.

9. A process according to claim 1, wherein the at least one vinyl heterocycle is selected from N-vinyllactam or at least one N-vinylamidine or a mixture thereof.

10. A process according to claim 9, wherein the N-vinyllactam is selected from N-vinylpyrrolidone, N-vinylpiperidone, N-vinylcaprolactam or a mixture thereof.

11. A process according to claim 9, wherein the N-vinylamidine is selected from N-vinylimidazole, N-vinyl-2-methylimidazole, N-vinyl-4-methylimidazole or a mixture thereof.

12. A process according to claim 1, the water-insoluble polymer comprising
a) from 0.1 to 99.9% by weight of N-vinylpyrrolidone as the at least one vinyl heterocycle
b) from 0.1 to 10% by weight of at least one difunctional crosslinking component
c) from 0 to 99.8% by weight of styrene or at least one monounsaturated styrene derivative or a mixture thereof,
the stated percentages by weight of the individual components a), b) and c) summing to 100%, being used.

13. A process according to claim 2, the water-insoluble polymer consisting essentially of
100% by weight of at least one N-vinylamide of an aliphatic carboxylic acid, or of the monomers obtained by partial or complete hydrolysis of said amido group and of the formula H₂C═CR²NR³C(O)R¹, where R¹, R² and R³, independently of one another, are hydrogen, C₁₋₂₀-alkyl, C₁₋₂₀-aryl or C₁₋₂₀-alkylaryl, being used.

14. A process according to claim 2, the water-insoluble polymer consisting essentially of
100% by weight of at least one vinylcarboxylic acid or esters, amides or salts thereof or of the monomers obtained by partial or complete hydrolysis of said amido group or ester group being used.

15. A process according to claim 13, wherein the N-vinylamide is selected from N-vinylformamide, N-vinylacetamide, N-vinyl-N-methylacetamide, N-vinyl-N-methylformamide, acyclic amidines or mixtures thereof.

16. A process according to claim 14, wherein the vinylcarboxylic acid or the esters, amides or salts thereof said vinylcarboxylic acid are selected from the group consisting of acrylic acid, acrylamide, C₁-C₄-alkyl acrylates, alkali metal acrylate, alkaline earth metal acrylate, methacrylic acid, methacrylamide, N-isopropylmethacrylamide, C₁-C₄-alkyl methacrylates, alkali metal methacrylate, alkaline earth metal methacrylate, maleic acid, maleic anhydride, maleimide, mono(C₁-C₄-alkyl)maleates, di(C₁-C₄-alkyl)maleates, monoalkali metal maleate, dialkali metal maleate, fumaric acid, fumaric acid monoamide, fumaric acid diamide, mono(C₁-C₄-alkyl)fumarate, di(C₁-C₄-alkyl)fumarate, monoalkali metal fumarate, dialkali metal fumarate and mixtures thereof.

17. A process according to claim 1, wherein the preparation is conducted in the presence of a polymer mixture which consists essentially of
a) a thermoplastic polymer and
b) water-insoluble polymers of vinyl heterocycles or N-vinylamides, it being possible for the N-vinylamide polymers to be partly or completely hydrolyzed to amines.

18. A process according to claim 1, wherein the preparation is conducted in the presence of a polymer mixture consisting essentially of
i) from 5 to 95% by weight of at least one styrene polymer and
ii) from 5 to 95% by weight of a crosslinked or uncrosslinked poly-N-vinylpyrrolidone.

19. A process according to claim 2, wherein the preparation is conducted in the presence of a polymer mixture consisting essentially of
i) from 5 to 95% by weight of at least one styrene polymer and
iii) from 5 to 95% by weight of a poly-N-vinylformamide or of a polyamine obtained by partial or complete hydrolysis of the poly-N-vinylformamide.

20. A process according to claim 1, wherein the preparation is conducted in the presence of a polymer mixture consisting essentially of
ii) from 5 to 95% by weight of a crosslinked or uncrosslinked poly-N-vinylpyrrolidone and
iii) from 5 to 95% by weight of a poly-N-vinylformamide or of a polyamine obtained by partial or complete hydrolysis of the poly-N-vinylformamide.

21. A process according to the preparation is conducted in the presence of a polymer mixture consisting essentially of
i) from 2 to 95% by weight of at least one styrene polymer,
ii) from 2 to 95% by weight of a crosslinked or uncrosslinked poly-N-vinylpyrrolidone and
iii) from 2 to 95% by weight of a poly-N-vinylformamide or of a polyamine obtained by partial or complete hydrolysis of the poly-N-vinylformamide.

22. A process according to claim 2, wherein the preparation is conducted in the presence of a polymer mixture which consists essentially of
a) a thermoplastic polymer and
b) water-insoluble polymers of vinyl heterocycles or N-vinylamides, it being possible for the N-vinylamide polymers to be partly or completely hydrolyzed to amines.

23. A process according to claim 1, wherein the preparation is conducted in the presence of a polymer mixture consisting essentially of
ii) from 5 to 95% by weight of a crosslinked or uncrosslinked poly-N-vinylpyrrolidone and
iii) from 5 to 95% by weight of a poly-N-vinylformamide or of a polyamine obtained by partial or complete hydrolysis of the poly-N-vinylformamide.

24. A process according to the preparation is conducted in the presence of a polymer mixture consisting essentially of
i) from 2 to 95% by weight of at least one styrene polymer,
ii) from 2 to 95% by weight of a crosslinked or uncrosslinked poly-N-vinylpyrrolidone and
iii) from 2 to 95% by weight of a poly-N-vinylformamide or of a polyamine obtained by partial or complete hydrolysis of the poly-N-vinylformamide.
